(19) United States
(12) Patent Application Publication
(10) Pub. No.: US 2011/0105585 A1
(43) Pub. Date: May 5, 2011

(54) METHODS FOR DIAGNOSING AND TREATING SQUAMOUS CELL CARCINOMA UTILIZING miRNA-205 AND INHIBITORS THEREOF

(75) Inventors: Robert M. Lavker, Chicago, IL (US); David G. Ryan, Chicago, IL (US); Jia Yu, Chicago, IL (US)

(73) Assignee: NORTHWESTERN UNIVERSITY, Evanston, IL (US)

(21) Appl. No.: 12/764,674

(22) Filed: Apr. 21, 2010

Related U.S. Application Data
(60) Provisional application No. 61/172,045, filed on Apr. 23, 2009.

(51) Int. Cl. A61K 31/7088; A61P 35/00; C12Q 1/68

(52) U.S. Cl. 514/44 A; 435/6

(57) ABSTRACT

Disclosed are diagnostic and therapeutic methods related to squamous cell carcinoma. In particular, the diagnostic methods relate to detecting miRNA-205, thereby diagnosing an aggressive form of squamous cell carcinoma. The therapeutic methods relate to inhibiting the function of miRNA-205, thereby treating an aggressive form of squamous cell carcinoma.

### CROSS-REFERENCE TO RELATED APPLICATIONS

The present application claims the benefit under 35 U.S.C. §119(e) to U.S. Provisional Patent Application No. 61/172,045, filed on Apr. 23, 2009, the content of which is incorporated by reference in its entirety.

### STATEMENT REGARDING FEDERALLY SPONSORED RESEARCH OR DEVELOPMENT

This invention was made with government support under Grant No. EY017536 awarded by the National Institutes of Health: National Eye Institute. The government has certain rights in the invention.

### BACKGROUND

The field of the invention relates to microRNAs (miRNAs) and the use of miRNAs and inhibitors of miRNAs in diagnostic and therapeutic methods. In particular, the field of the invention relates to miRNA-205 and the use of miRNA-205 and inhibitors of miRNA-205 in diagnostic and therapeutic methods for aggressive forms of squamous cell carcinoma.

MicroRNAs (miRNAs) are small, 20- to 24-nucleotide, noncoding RNAs found in diverse organisms. In animals, most miRNAs mediate posttranscriptional silencing by binding with partial complementarity to the 3′ UTR of the target mRNA (1, 2). These endogenous, silencing RNAs have been shown to play important roles in development and differentiation ( 3-6), cellular stress responses (7), and cancer (8-11).

The role of miRNAs in stratified squamous epithelia remains poorly understood. Inactivation of Dicer in mouse skin caused hair follicles to evaginate into the epidermis rather than invaginating downward, thus forming cyst-like structures (12, 13). These results underscore the importance of miRNAs in the regulation of epidermal and follicular development. miRNAs have also been extensively profiled in the corneal epithelium and show expression patterns that are regionally restricted (14). For example, miR-184 was the most abundant miRNA in the corneal epithelium; however, it was conspicuously absent from the limbal epithelium, an area enriched in corneal epithelial stem cells (15-18). In contrast, miR-205 is broadly expressed throughout all viable cell layers in nearly all stratified squamous epithelia including the corneal, limbal, and conjunctival epithelia of the eye (12, 14). Thus, the corneal epithelium is unique in that it exhibits distinct as well as overlapping expression of miR-184 and miR-205 (14).

miRNAs have been predicted to regulate thousands of mammalian genes (19); however, few targets have been experimentally validated for the great majority of these miRNAs. With the exception of a recent demonstration that a p63-related family member is negatively regulated by miR-203 (20), little is known about stratified squamous epithelial miRNA targets. We report that miR-205 represses SH2-containing phosphoinositide 5′-phosphatase 2 (SHIP2). We also find that miR-184 negatively modulates the activity of miR-205 to maintain SHIP2 levels. This finding is the first demonstration that a miRNA can interfere with another miRNA to ensure the expression of a target protein. We show: (i) that SHIP2 levels can be modulated in a variety of epithelial cells using gain- and loss-of-function experiments with miR-184 and miR-205 and (ii) that manipulating SHIP2 levels through miRNAs diminishes Akt signaling leading to decreased keratinocyte survival. Finally, we find a reciprocal relationship between miR-205 and SHIP2 expression in squamous cell carcinoma (SCC) cell lines and suggest that miR-205 may be viewed as a tumor promoter in the context of SCCs.

### SUMMARY

Disclosed are methods for utilizing miRNA-205 and inhibitors of miRNA-205 for diagnosing and treating squamous cell carcinoma, in particular, aggressive forms of squamous cell carcinoma. In some embodiments, the disclosed methods may be diagnostic. For example, the disclosed methods may be utilized to diagnose an aggressive form of squamous cell carcinoma in a patient having squamous cell carcinoma. The methods may include detecting a level of miRNA-205 in squamous carcinoma cells of the patient where the detected level of miRNA-205 in the squamous carcinoma cells of the patient is characteristic of the aggressive form of squamous cell carcinoma, thereby diagnosing the aggressive form of squamous cell carcinoma in the patient. In further embodiments, the methods may include detecting a level of control RNA in the squamous carcinoma cells of the patient and comparing the detected level of miRNA-205 in the squamous carcinoma cells of the patient to the detected level of the control RNA in the squamous carcinoma cells of the patient. A ratio of the detected miRNA-205 to the detected level of control RNA may be calculated, where the ratio is characteristic of the aggressive form of squamous cell carcinoma, thereby diagnosing the aggressive form of squamous cell carcinoma in the patient.

In the methods, miRNA-205 may be detected by obtaining a nucleic acid sample from the squamous carcinoma cells of the patient and contacting the sample with a probe that binds to miRNA-205. Suitable probes may include DNA probes or RNA probes that hybridize to miRNA-205. The probe optionally may be modified, e.g., with a label for detection. In the methods, miRNA-205 may be detected by performing assays known in the art (e.g., Northern blots). Preferably, miRNA-205 is detected utilizing a solution hybridization assay (e.g., an RNase protection assay). Other methods for detecting miRNA-205 may include, but are not limited to, methods for detecting miRNA as known in the art (54).

The methods contemplated herein also may include methods for treating or preventing an aggressive form of squamous cell carcinoma in a patient in need thereof where the methods include administering to the patient an inhibitor of miRNA-205. Suitable inhibitors of miRNA-205 may include antagomirs. In further embodiments, inhibitors of miRNA-205 may include nucleic acid molecules that compete with miRNA-205 for a target nucleic acid molecule, such as miRNA-184, which competes for SHIP-2 mRNA as a target. The inhibitor may be administered as part of pharmaceutical composition. In some embodiments, the inhibitor is administered via expression from an ectopic vector.

The aggressive forms of squamous cell carcinoma diagnosed, treated, or prevented by the methods disclosed herein may be defined by clinical criteria. For example, aggressive forms of squamous cell carcinoma may include but are not limited to forms that exhibit rapid growth (e.g., where the squamous cell carcinoma forms a tumor that doubles in size over a period of less than about six (6) months), large size (e.g., where the squamous cell carcinoma forms a tumor having a size greater than about 1.5 cm), recurrence, and metastasis or invasiveness.

Also contemplated herein are methods for modulating expression of SHIP-2 expression in a cell. For example, the methods may include increasing expression of SHIP-2 in a cell (e.g., a squamous cancer cell) by introducing to the cell an inhibitor of miRNA-205. Suitable inhibitors may include antagomirs of miRNA-205 or competitors of miRNA-205 (e.g., miRNA-184) as disclosed herein.

### BRIEF DESCRIPTION OF THE FIGURES

FIG. 1. miR-205 targets SHIP2 at 3′ UTR and can be regulated by miR-184. (A) Sequence of the miR-205 and miR-184 binding sites within the human SHIP2 (INPPL1) 3′ UTR. Shaded areas represent conserved complementary nucleotides of miR-184 and miR-205 seed sequences in various mammals (H.s, human; M.m, mouse; R.n, rat; C.f, chicken). (B) Schematic of the reporter constructs showing entire 3′ UTR SHIP2 sequence (SHIP2_wt) and the mutated 3′ UTR nucleotides of the miR-205 binding site (SHIP2_mut1, shaded nucleotide sequence). SHIP2_mut2 represents the reporter construct containing mutated overlapping nucleotides of miR-184 and miR-205 (shaded nucleotide sequence). SHIP2_mut3 represents the reporter construct containing nucleotides predicted to be exclusively used for miR-184 binding to SHIP2 mRNA (shaded nucleotide sequence). (C) Luciferase activity of (i) SHIP2_wt in the presence of 10 nM of miR-205 showing the inhibitory activity of this reporter and (ii) the SHIP2_mut1 and mut2 reporters, showing that miR-205 mimic cannot inhibit the luciferase activity of these constructs compared with the wild-type construct. Error bars (SEM) are derived from six experiments in triplicate. (D) Luciferase activity of SHIP2_wt reporter in the presence (+) or absence (−) of various concentrations of miR-205, miR-184, or nontargeting (irrelevant) mimics. Error bars (SEM) are derived from three experiments in triplicate. (E) Luciferase activity of SHIP2_mut3 reporter showing that (i) this mutation does not inhibit miR-205 binding to SHIP2 3′ UTR; (ii) miR-184 does not inhibit this mutated reporter; and (iii) cotransfection of miR-184 and miR-205 cannot restore luciferase activity of 184 mut3. Error bars (SEM) are derived from three experiments in triplicate. Controls for these experiments are shown in FIG. 6C and D. (F) Luciferase activity of SHIP2_wt and SHIP2_mut1 in HEKs showing that endogenous miR-205 inhibits SHIP2. Positive controls (184/205_PER) are shown in FIG. 6E.

FIG. 2. SHIP2 levels are controlled by miR-205 and miR-184. (A) Immunoblotting of SHIP2 in HeLa cells that were treated with a miR-205 mimic, decrease protein 48 and 72 h after treatment. (B) Immunofluorescence microscopy of HeLa cells stained with anti-SHIP2 and anti-SHIP2/DAPI showing a marked decrease in staining 72 h after treatment with miR-205 mimic. Staining data at 48 h is presented in FIG. 7A. (C) Immunoblotting of SHIP2 in HeLa cells that were untreated (1), transfected with an irrelevant mimic (ir-mim; 2), miR-205 mimic (205-mim; 3), miR-205 mimic plus and irrelevant mimic (ir+205-mim; 4), miR-184 mimic plus an irrelevant mimic (ir+184-mim; 5), miR-184 plus miR-205 mimics (184+205-mim;6), and miR-184 mimic (184-mim; 7) for 48 h. miR-205 mimic reduces SHIP2 levels (3, 4) whereas miR-184 inhibits miR-205 from reducing SHIP2 levels (6). (D) Northern analysis using a miR-205 specific probe showing a marked decrease in miR-205 levels in HEKs treated with an antagomir to miR-205 (Antago-205) for 48 and 72 h. (E) Immunofluorescence microscopy of HEKs stained with SHIP2 showing an increase in staining after 72 h of treatment with Antago-205. Staining data at 48 h are presented in FIG. 7B. Numbers below the panels represent the normalized expression signal of proteins and RNAs.

FIG. 3. miR-205 affects the Akt pathway in keratinocytes directly through targeting of SHIP2 and is inversely correlated with SHIP2 in SCC cell lines. (A) Immunoblotting of SHIP2, phosphorylated Akt (p-Akt), total pan (1/2/5) Akt, phosphorylated BAD, total BAD, phosphorylated PTEN (p-PTEN), and phosphorylated GSK3β (p-GSK3β) in HEKs that were untreated (un-rx) or treated with an ir-antagomir or Antago-205 for 48 h. α-Tubulin serves as a loading control. (B) Immunoblots of SHIP2, p-Akt, AKT, and α-tubulin in HEKs 72 h after transfection with SHIP2 siRNA and control siRNA, showing decreases in SHIP2 and increases in p-Akt. (C) Immunoblots of SHIP2, p-Akt, AKT, and α-tubulin in HEKs 48 h after treatment with an antagomir to miR-205 or an irrelevant antagomir. HEKs were subsequently treated for another 72 h with combinations of siRNA to SHIP2, control siRNA, antagomir-205, and irrelevant antagomir. (D) Keratinocytes were stained with propidium iodide and annexin V 48 h after treatment with an ir-antagomir or Antago-205 and compared with untreated cells. Late apoptotic cells are seen in the top right quandrant. (E) Northern analysis of oral SCC cell lines using a miR-205-specific probe showing increases in miR-205 in SCC68 and CAL27 cells. (F) Northern analysis with a miR-205-specific probe in SCC68 cells that were treated with an ir-antagomir or Antago-205 for 48 h. U6 serves as a loading control. (G) Immunoblotting of SHIP2, p-Akt, total Akt, p-PTEN, p-GSK3β, p-BAD, and BAD in SCC68 cells treated as described in F. α-Tubulin serves as loading control. (H) SCC68 cells were treated as described in F and G and then stained with propidium iodide and annexin V. Numbers below the panels represent the normalized expression signal of proteins and RNAs.

FIG. 4. miR-184 alters the ability of miR-205 to affect SHIP2 in corneal keratinocytes in vitro and in vivo. (A) Northern analysis of primary human corneal epithelial (HCEKs) cells using specific probes for miR-184 and miR-205, showing expression of both of these miRNAs in untreated and control (Ir-antagomir) cells. U6 serves as a loading control. Shown is immunoblotting of SHIP2 and α-tubulin in HCEKs that were untreated or were treated with Ir-antagomir, Antago-205, or an antagomir to mi R-184 (Antago-184) for 72 h. (B) Immunofluorescence microscopy of HCEKs stained for SHIP2 showing a marked decrease in staining after a 72-h treatment with antagomir-184, whereas treatment with antagomir to miR-205 resulted in an increase in SHIP2 staining. (C and D) Serial frozen sections of human limbal and corneal epithelium immunohistochemically stained with an antibody that recognizes IgG (control, C) or SHIP2 (D). (E and F) Higher magnification of the boxed areas of the limbal (l, E) and corneal (c, F) epithelia, showing a decrease in SHIP2 staining in the limbal epithelium compared with the corneal epithelium. Numbers below the panels represent the normalized expression signal of proteins and RNAs.

FIG. 5. Proposed regulatory effects of miR-205 and miR-184 on SHIP2 levels in various epithelial contexts. (A) Epidermal keratinocytes. Decreasing miR-205 via antagomir-205 increases SHIP2 levels resulting in the dampening of Akt signaling and an increase in apoptosis and cell death. (B) Corneal keratinocytes. Decreasing miR-184 via antagomir-184 “releases” miR-205 to reduce SHIP2 levels augmenting the Akt pathway, with increased cell survival and angiogenesis as possible outcomes. (C) SCC. Ectopic expression of miR-184 or treatment with an antagomir to miR-205 represents potential therapeutic modalities for the treatment of SCCs by increasing SHIP2 levels, which might act as a tumor suppressor in these neoplasias.

FIG. 6. Luciferase reporter assays showing effects of miR-184 and miR-205 on SHIP2 levels. (A) Luciferase activity of SHIP2 reporter in the presence of various concentrations (1-100 nM) of miR-184, showing that this miRNA cannot inhibit the luciferase activity of this construct. Error bars (SEM) are derived from three experiments in triplicate. (B) Luciferase activity of SHIP2 reporter in the presence of various concentrations (1-100 nM) of miR-205, showing the inhibitory activity of this reporter. Error bars (SEM) are derived from six experiments in triplicate. (C) Luciferase activity of mutated SHIP2 reporter (SHIP-mut3) cotransfected with either an irrelevant mimic (ir-mim+SHIP2_mut 3) or a miR-184 mimic (184+SHIP2_mut 3), showing that cotransfections do not affect luciferase activity. Error bars (SEM) are derived from three experiments in triplicate. (D) Luciferase activity of mutated SHIP2 reporter (SHIP2_mut3) showing that cotransfection with either miR-184 mimic and miR-205 mimic (205+184 SHIP2_mut3) or miR-205 mimic and an irrelevant mimic (205+ir-mim+SHIP2_mut3) fail to restore luciferase activity of SHIP2 mutation 3; conversely, cotransfection of miR-l84 mimic and an irrelevant mimic (184+ir-mim+SHIP_mut3) does not affect luciferase activity. Error bars (SEM) are derived from three experiments in triplicate. (E) Luciferase activity of 184/205_PER and empty reporters in HEKs showing that endogenous miR-205 negatively regulates the positive control.

FIG. 7. (A) Immunofluorescence microscopy of HeLa cells stained with anti-SHIP2 and anti-SHIP2/DAPI showing a marked decrease in staining after 48 and 72 h of treatment with miR-205 mimic, compared with untreated cells and cells treated with an irrelevant mimic (ir-mim). (B) immunofluorescence microscopy of HEKs stained with SHIP2 showing an increase in staining after 48 and 72 h of treatment with an antagomir to miR-205 (Antago-205), compared with untreated cells and cells treated with an irrelevant mimic (ir-antagomir).

### DETAILED DESCRIPTION

The subject matter disclosed herein is described using several definitions, as set forth below and throughout the application.

Unless otherwise noted, the terms used herein are to be understood according to conventional usage by those of ordinary skill in the relevant art. In addition to the definitions of terms provided below, it is to be understood that as used in the specification, embodiments, and in the claims, “a”, “an”, and “the” can mean one or more, depending upon the context in which it is used.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” or “approximately” will mean up to plus or minus 10% of the particular term and “substantially” and “significantly” will mean more than plus or minus 10% of the particular term.

As used herein, the terms “patient” and “subject” may be used interchangeably and refer to one who receives medical care, attention or treatment. As used herein, the term is meant to encompass a person diagnosed with a disease such as squamous cell carcinoma or at risk for developing squamous cell carcinoma (e.g., a person who may be symptomatic for squamous cell carcinoma but who has not yet been diagnosed). As used herein, the term terms “patient” and “subject” are meant to encompass a person diagnosed with an aggressive form of squamous cell carcinoma or at risk for developing an aggressive form of squamous cell carcinoma. As used herein, an “aggressive” form of squamous cell carcinoma may be defined by several clinical criteria, which include, but are not limited to rapid growth (e.g., the tumor mass comprising the squamous cell carcinoma doubling in size in as few as several months (e.g., as few as six(6) months)), large size (e.g., the tumor mass comprising the squamous cell carcinoma having a diameter of at least 1.5 cm), a history of recurrence, and metastasis or invasiveness.

As used herein the terms “diagnose” or “diagnosis” or “diagnosing” refer to distinguishing or identifying a disease, syndrome or condition or distinguishing or identifying a person having a particular disease, syndrome or condition. As used herein the terms “prognose” or “prognosis” or “prognosing” refer to predicting an outcome of a disease, syndrome or condition. The methods contemplated herein include diagnosing an aggressive form of squamous cell carcinoma in a patient (e.g. in a patient having squamous cell carcinoma). The methods contemplated herein also include determining a prognosing for a patient having squamous cell carcinoma (e.g., by determining a level of miRNA-205 in squamous cancer cells of the patient).

In some embodiments of the methods disclosed herein, miRNA-205 may be detected utilizing methods for detecting miRNA as known in the art. (See, e.g., Hunt et al. (54), the content of which is incorporated herein by reference in its entirety.) For example, miRNA-205 may be detected by obtaining a nucleic acid sample from the squamous carcinoma cells of the patient and contacting the sample with a probe that binds to miRNA-205. Suitable probes may include DNA probes or RNA probes that hybridize to miRNA-205. The probe optionally may be modified, e.g., with a label for detection. In the methods, miRNA-205 may be detected by performing assays known in the art (e.g., Northern blots). Preferably, miRNA-205 is detected utilizing a solution hybridization assay (e.g., an RNase protection assay).

As used herein, the term “treatment,” “treating,” or “treat” refers to care by procedures or application that are intended to relieve illness or injury. Although it is preferred that treating a condition or disease such as a squamous cell carcinoma will result in an improvement of the condition, the term treating as used herein does not indicate, imply, or require that the procedures or applications are at all successful in ameliorating symptoms associated with any particular condition. Treating a patient may result in adverse side effects or even a worsening of the condition which the treatment was intended to improve.

Treating as contemplated herein may include administering to a patient an inhibitor of miRNA-205. The term “inhibitor” as used herein refers to any molecule, substance, or drug that when properly administered, decreases, downwardly modulates, or prohibits a reaction or an activity. An inhibitor of miRNA-205 may include a nucleic acid molecule which prevents miRNA-205 from hybridizing to a target of miRNA-205 (e.g., SHIP-2 mRNA, in particular within the 3′ untranslated region of SHIP-2 mRNA). An inhibitor of miRNA-205 may include a nucleic acid that hybridizes with miRNA-205 or which hybridizes to a target of miRNA-205 as a competitor (e.g., miRNA-184). An inhibitor of miRNA-205 may include a chemically modified nucleic acid such as an “antagomir.” Antagomirs are known in the art. (See, e.g., U.S. Published Application Nos. 2007-0123482 and 2007-0213292, which contents are incorporated herein by reference in their entireties).

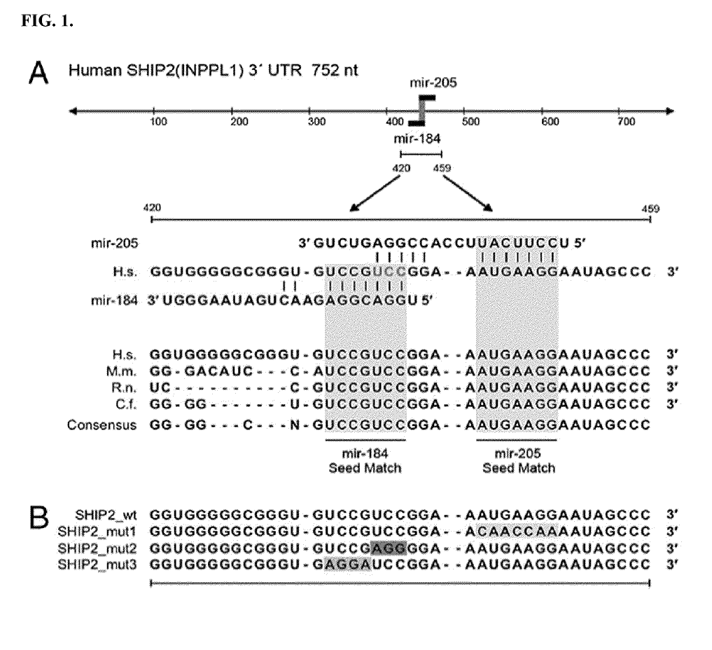

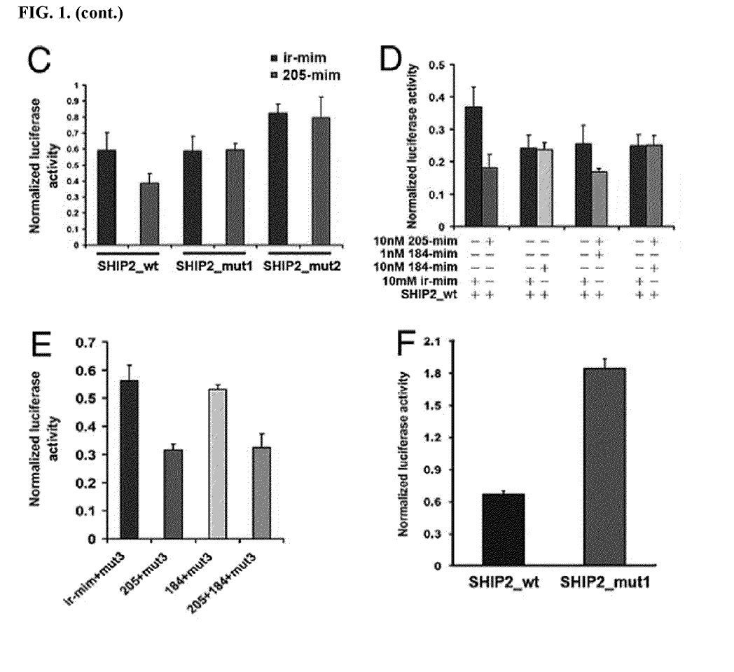

As used herein, “miRNA-205” refers to a miRNA molecule that is twenty-two (22) nucleotides in length and has the sequence 5′-UCCUUCAUUCCACCGGAGUCUG-3′ (SEQ ID NO:1), and “miRNA-184” refers to a miRNA molecule that is that is twenty-two (22) nucleotides in length and has the sequence 5′-UGGACGGAGAACUGAUAAGGGU-3′ (SEQ ID NO:2). As disclosed herein, miRNA-205 and miRNA-184 may hybridize (e.g., competitively) to a region of the 3′ untranslated region (UTR) of the mRNA for SH2-containing phosphoinositide 5′-phosphatase 2 (SHIP2) (SEQ ID NO:3), otherwise referred to as inositol polyphosphate phosphatase-like 1 (INPPL1). (See National Center for Biotechnology Information (NCBI) Reference Sequence: NM_{—}001567.2, providing the corresponding cDNA sequence of SHIP2 mRNA). The sequence of the 3′ UTR of SHIP2 mRNA to which miRNA-205 and miRNA-184 hybridize includes SEQ ID NO:4. (See FIG. 1A).

The term “nucleic acid” or “nucleic acid sequence” refers to a nucleotide, oligonucleotide, polynucleotide, or fragments or portions thereof, which may be single or double stranded, and represent the sense or antisense strand. A nucleic acid may include RNA or DNA, and may be of natural or synthetic origin. For example, a nucleic acid may include mRNA or cDNA. The terms “oligonucleotide” and “polynucleotide” may be utilized interchangeably herein. These phrases also refer to RNA or DNA of genomic or synthetic origin which may be single-stranded or double-stranded and may represent the sense or the antisense strand, to peptide nucleic acid (PNA), or to any RNA-like or DNA-like material.

An oligonucleotide may include an RNA or DNA molecule that has a sequence of bases on a backbone which are arranged in such a way that they can enter into a bond with a nucleic acid having a sequence of bases that are complementary to the bases of the oligonucleotide (i.e., a target nucleic acid as discussed herein). The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides also may include derivatives, in which the hydrogen of the hydroxyl group is replaced with organic groups (e.g., an allyl group). Oligonucleotides of the method which function as probes generally are at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including chemical synthesis. The oligonucleotide may be modified. For example, the oligonucleotide may be labeled with an agent that produces a detectable signal (e.g., a fluorophore). In other embodiments, the oligonucleotide may be conjugated to a lipid molecule (e.g., cholesterol).

A “probe” refers to an oligonucleotide that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect a nucleic acid sequence by virtue of the sequence characteristics of the target. Probes can be labeled or unlabeled, or modified in any of a number of ways well known in the art. A probe may specifically hybridize to a target nucleic acid.

A “target nucleic acid” refers to a nucleic acid molecule containing a sequence that has at least partial complementarity with a probe oligonucleotide. A probe may specifically hybridize to a target nucleic acid. As contemplated herein, “target nucleic acids” may include miRNA-205 and SHIP2 mRNA, and in particular regions of the 3′ UTR of SHIP2 mRNA. (See, e.g., SEQ ID NO:4).

An oligonucleotide (e.g., a probe) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art. Oligonucleotides used as probes for specifically detecting (i.e., detecting a particular target nucleic acid sequence) a target nucleic acid generally are capable of specifically hybridizing to the target nucleic acid.

“Homology” refers to sequence similarity or, interchangeably, sequence identity, between two or more polynucleotide sequences. Homology, sequence similarity, and percentage sequence identity may be determined using methods in the art and described herein. The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed below). Percent identity may be measured over the length of an entire defined sequence, for example, as defined by a particular SEQ ID number.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

The words “insertion” and “addition” refer to changes in a nucleotide sequence resulting in the addition of one or more nucleotides. For example, an insertion or addition may refer to 1, 2, 3, 4, 5, or more nucleotides.

“Operably linked” refers to the situation in which a first nucleic acid sequence is placed in a functional relationship with a second nucleic acid sequence. For instance, a promoter is operably linked to a gene for a miRNA if the promoter affects the transcription or expression of the miRNA. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2^{nd} ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The disclosed methods may include obtaining a sample of nucleic acid from a patient (e.g., a nucleic acid sample from squamous carcinoma cells). Numerous methods are known in the art for isolating total nucleic acid (e.g., RNA) from a patient sample. Previously described methods, kits or systems for extraction of mammalian RNA or viral RNA may be adapted, either as published or modified for the extraction of tumor-derived or associated RNA. For example, Roche MagNA Pure RNA extraction system and methods (Roche Diagnostics, Roche Molecular Systems, Inc., Alameda, Calif.), may be used. Or, methods described in U.S. Pat. No. 6,916,634 may also be employed. Additional examples of RNA extraction are described below.

“Substantially isolated or purified” nucleic acid is contemplated herein. The term “substantially isolated or purified” refers to nucleic sequences that are removed from their natural environment, and are at least 60% free, preferably at least 75% free, and more preferably at least 90% free, even more preferably at least 95% free from other components with which they are naturally associated.

As used herein, the term “assay” or “assaying” means qualitative or quantitative analysis or testing. The methods contemplated herein may include assaying miRNA-205 in squamous cancer cells of a patient in order to determine a level of miRNA-205 in the squamous cancer cells in the patient.

As used herein the term “ratio” refers to the relation in degree or number between two similar things. For example, the methods contemplated herein may include determining the relative amount of miRNA-205 to a control RNA in a sample from a patient having squamous cell carcinoma. As such, a ratio of the amount of miRNA-205 to the amount of the control RNA may be determined in the methods for providing a diagnosis of an aggressive form of squamous cell carcinoma.

“Transformation” describes a process by which exogenous RNA or DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted RNA or DNA is capable of replication either as part of an episomal nucleic acid or as part of the host chromosome, as well as transiently transformed cells which express the inserted RNA or DNA for limited periods of time.

As used herein, the term “transfection” means the transfer of exogenous nucleic acid into a cell. Transfection methods may include physical methods and biological methods. Transfection may include transduction (e.g., by infection with a viral vector) and electroporation via exposing a cell to an electric current. Methods of cell transfection also may include CaCl₂, CaPO₄, and liposome-mediated transfection. Other methods for introducing DNA into cells may include nuclear microinjection or polycation-, polybrene-, or polyormithine-mediated transfection.

A “composition comprising a given polynucleotide sequence” refer broadly to any composition containing the given polynucleotide sequence. The composition may comprise a dry formulation or an aqueous solution. The compositions may be stored in any suitable form including, but not limited to, freeze-dried form and may be associated with a stabilizing agent such as a carbohydrate. The compositions may be aqueous solution containing salts (e.g., NaCl), detergents (e.g., sodium dodecyl sulfate; SDS), and other components (e.g., Denhardt's solution, dry milk, salmon sperm DNA, and the like).

Pharmaceutical compositions comprising inhibitors of miRNA-205 are contemplated herein. In some embodiments, the pharmaceutical compositions may include a therapeutically effective amount of an inhibitor of miRNA-205 and one or more pharmaceutically acceptable carriers, excipients, or diluents (i.e., agents), which are nontoxic to the cell or mammal being exposed thereto at the dosages and concentrations employed. Often a physiologically acceptable agent is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

### EXAMPLE

The following example is illustrative and are not intended to limit the disclosed subject matter. Reference is made to Yu et al., “MicroRNA-184 antagonizes microRNA-205 to maintain SHIP2 levels in epithelia,” PNAS (December 9, 2008) 105(49):19300-19305, the content of which is incorporated herein by reference in its entirety.

Abstract

Despite their potential to regulate approximately one-third of the whole genome, relatively few microRNA (miRNA) targets have been experimentally validated, particularly in stratified squamous epithelia. Here we demonstrate not only that the lipid phosphatase SHIP2 is a target of miRNA-205 (miR-205) in epithelial cells, but, more importantly, that the corneal epithelial-specific miR-184 can interfere with the ability of miR-205 to suppress SHIP2 levels. This is the first example of a miRNA negatively regulating another to maintain levels of a target protein. Interfering with miR-205 function by using a synthetic antagomir, or by the ectopic expression of miR-184, leads to a coordinated damping of the Akt signaling pathway via SHIP2 induction. This was associated with a marked increase in keratinocyte apoptosis and cell death. Aggressive squamous cell carcinoma (SCC) cells exhibited elevated levels of miR-205. This was associated with a concomitant reduction in SHIP2 levels. Partial knockdown of endogenous miR-205 in SCCs markedly decreased phosphorylated Akt and phosphorylated BAD levels and increased apoptosis. We were able to increase SHIP2 levels in SCC cells after inhibition of miR-205. Therefore, miR-205 might have diagnostic value in determining the aggressivity of SCCs. Blockage of miR-205 activity with an antagomir or via ectopic expression of miR-184 could be novel therapeutic approaches for treating aggressive SCCs.

Results

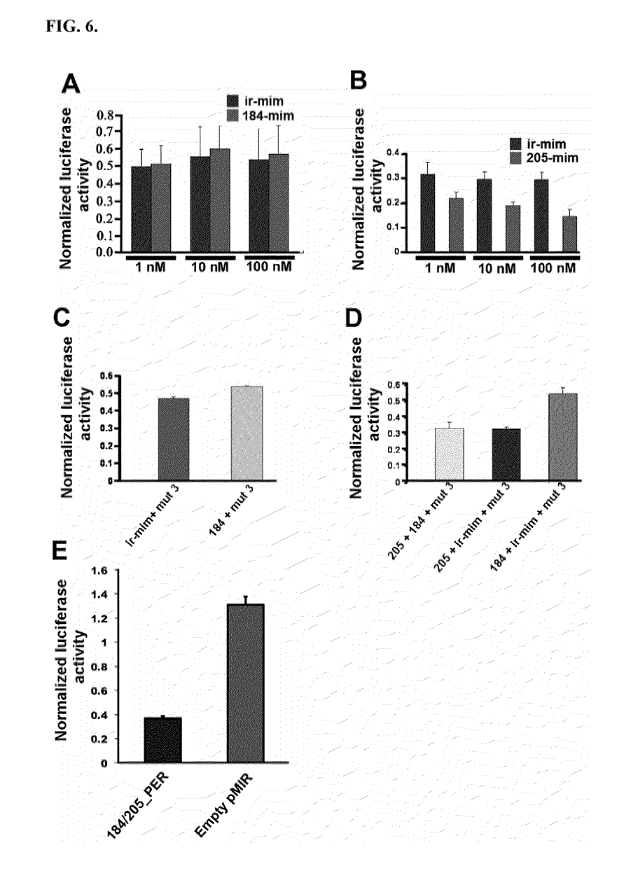

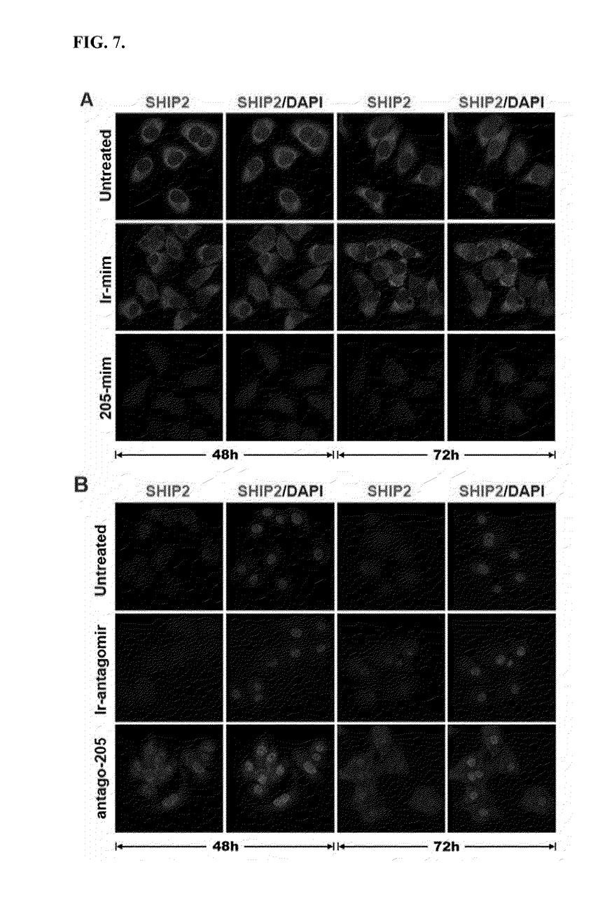

miR-205 Targets SHIP2. We found miR-205 in all squamous epithelium that we examined (14). We also reported that miR-184 and miR-205 are the most abundant miRNAs in corneal epithelium and that miR-184 expression was restricted to the corneal epithelium (14). Bioinformatic analysis suggested that, in humans, the SHIP2 (Inppl1) 3′ UTR is a putative target of both miR-184 and miR-205 (21) and is the only gene with overlapping binding sites to these two miRNAs. The overlapping nucleotide sequence including the trinucleotide sequence AGG. To test this prediction (FIG. 1A), we cotransfected HeLa cells with a miR-184 or miR-205 mimic and luciferase reporter constructs carrying the entire 3′ UTR of SHIP2 mRNA (FIG. 1B). In cells treated with a miR-205 mimic, we found a marked reduction (≈50%) in luciferase activity (FIGS. 1C and D and 6B; however, no reduction in luciferase activity was seen in transfectants expressing miR-184 (FIG. 6A and FIG. 1D), suggesting that miR-184 does not inhibit SHIP2. To confirm this result, we mutated the miR-205 binding site on SHIP2 3′ UTR (FIG. 1B, SHIP2_mut1). The mutation prevented miR-205 from interfering with luciferase activity, indicating that the 3′ UTR of SHIP2 is indeed a target of miR-205 (FIG. 1C).

In an effort to confirm that endogenous miR-205 regulates SHIP2 expression, we transfected SHIP2_wt or SHIP2_mut1 reporters into primary human epidermal keratinocytes (HEKs), respectively. Endogenous miR-205 indeed inhibited the luciferase activity of the SHIP2_wt but did not affect the luciferase activity of the SHIP2_mut I (FIG. 1F).

miR-184 Negatively Interferes with the Regulation of SHIP2 by miR-205. Interestingly, when we cotransfected equal amounts of miR-184 and miR-205 into HeLa cells, miR-205 no longer inhibited luciferase activity of the SHIP2 reporter (FIG. 1D). This suggested that the binding of miR-184 through its seed sequence (the nucleotides on a miRNA that interact with a target) prevents full binding of miR-205 with its complementary nucleotides and that nucleotides upstream of the miR-205 seed match (the complementary nucleotides of the target) on SHIP2 3′ UTR are required for full miR-205 activity (FIG. 1B). To confirm this, we mutated the 3 nucleotides upstream of the seed match predicted for full binding of miR-205. Cotransfection of this mutated construct with a miR-205 mimic did not decrease luciferase activity (FIG. 1C SHIP2_mut2), indicative that these nucleotides are required for miR-205 binding.

The observation that miR-184 interfered with the ability of miR-205 to regulate SHIP2 levels can most easily be explained by a competition for binding to the 3′ UTR. To test this idea, we mutated the nucleotides predicted to be exclusively used for miR-184 binding to SHIP2 mRNA (FIG. 1A and B, SHIP2_mut3). miR-205 mimic was still able to suppress luciferase activity when cotransfected with SHIP2_mut3 (FIG. 1E, blue and red columns), indicative that this mutation did not affect the overall inhibitory activity of miR-205. Cotransfection of SHIP2_mut3 with miR-184 mimic had no effect on luciferase activity (FIG. 1E, gray column, and FIG. 6C), confirming that miR-184 does not directly inhibit SHIP2. However, when we cotransfected miR-205 plus miR-184 mimic with SHIP2_mut3, the luciferase activity was reduced by ≈60% (FIG. 1E, orange column, and FIG. 6D). This provided additional data in support of the idea that miR-184 negatively regulates miR-205 to maintain SHIP2 levels in HeLa cells.

Summarizing these results, mir-205 and mir-184 include an overlapping trinucleotide sequence, “AGG,” which appears to be required for miR-205 binding to SHIP2 3′ UTR as does the seed sequence of miR-205. Transfection of miR-205 mimic inhibited luciferase activity, whereas transfection of miR-184 mimic had no effect. Cotransfection of 1 nM miR-184 and 10 nM miR-205 mimics did not completely restore luciferase activity, whereas cotransfection of equal amounts of miR-184 and miR-205 mimics completely rescued luciferase activity.

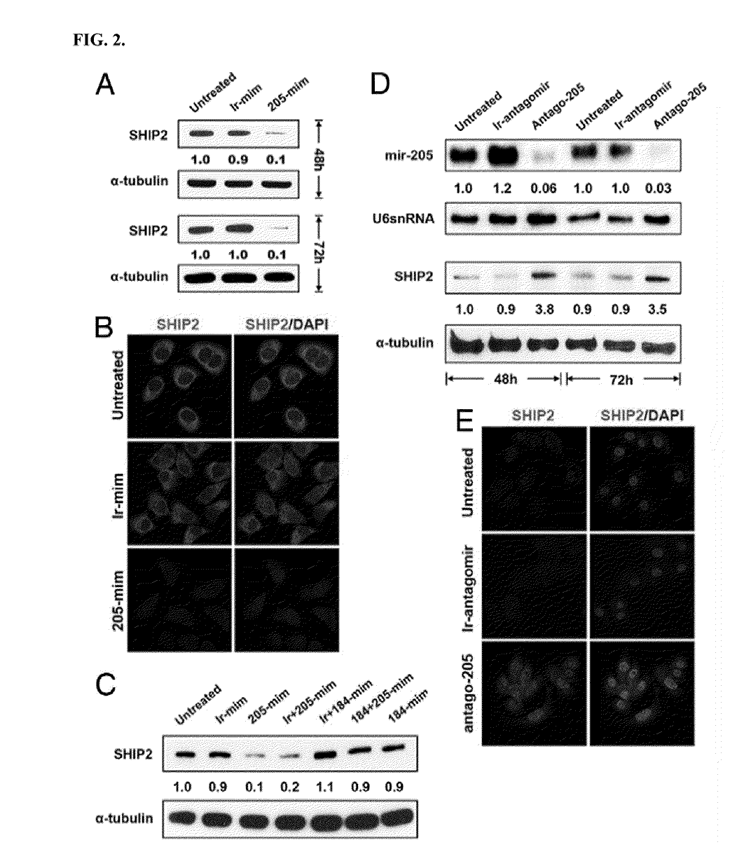

SHIP2 Protein Is Diminished by miR-205. HeLa cells have negligible endogenous levels of miR-205 (22) and readily detectable levels of SHIP2 (23). The most straightforward prediction from our luciferase reporter assays would be that ectopic expression of miR-205 should reduce SHIP2 protein levels in HeLa cells. We found that treatment of HeLa cells with the miR-205 mimic indeed caused a marked reduction in SHIP2 expression, whereas treatment with an irrelevant (nontargeting) mimic caused no reduction in SHIP2 protein (FIG. 2A). Similarly, SHIP2 immunoreactivity was diminished after HeLa cells were transfected with the miR-205 mimic when compared with untreated HeLa cells or cells treated with the irrelevant mimic (FIG. 2B). Taken together, these findings indicate that, in HeLa cells, SHIP2 can be negatively regulated by miR-205.

We next considered whether miR-184 had the capacity to maintain SHIP2 expression by antagonizing miR-205. If this was the case, we would expect to see an increase in endogenous SHIP2 protein after transfection with mimics to miR-184 and miR-205. As demonstrated previously, transfection of HeLa cells with miR-205 mimic led to a marked reduction in SHIP2 (FIG. 2A and C, lane 3). In contrast, treatment with a miR-184 mimic (FIG. 2C, lane 7), a miR-184 mimic plus an irrelevant mimic (FIG. 2C, lane 5), or a miR-184 mimic plus a miR-205 mimic (FIG. 2C, lane 6) did not reduce the SHIP2 levels. These findings confirm the luciferase reporter data indicating that miR-184 blocks the ability of miR-205 to negatively regulate SHIP2.

To study this novel regulation of SHIP2 in squamous epithelia, we first used primary HEK cultures. These cells express miR-205 but do not express miR-184, thereby making the analysis of SHIP2 more straightforward. We reasoned that down-regulation of miR-205 should result in a rise in SHIP2 levels. We conducted such a miRNA loss-of-function study using an antagomir to miR-205 (Antago-205). Antagomirs are cholesterol-linked single-stranded RNAs that are complementary to a specific miRNA and cause the depletion of the miRNA (24). Endogenous miR-205 was markedly reduced at 48 and 72 h after treatment with Antago-205, whereas an irrelevant antagomir (Antago-124—a neuronal-specific miRNA (25)) had no effect (FIG. 2D). As predicted, HEKs treated with Antago-205 showed a marked increase in SHIP2 levels by Western (FIG. 2D) and immunohistochemical (FIG. 2E) analyses when compared with the irrelevant antagomir-treated or untreated HEKs. Immunoblotting of SHIP2 and α-tubulin in HEKs showed an increase in SHIP2 expression 48 and 72 h after treatment with Antago-205. Thus, in HeLa cells and HEKs, SHIP2 levels are down-regulated by miR-205.

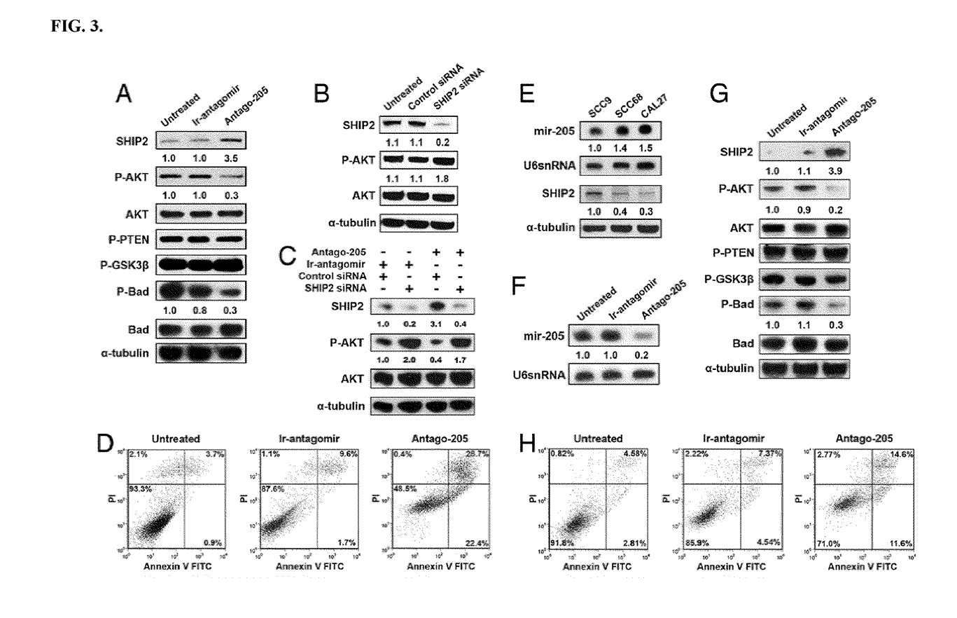

Down-Regulation of miR-205 Dampens Akt Signaling. One of the roles ascribed to SHIP2 has been the negative regulation of the Akt pathway (26-28); however, this ability of SHIP2 has not been investigated in keratinocytes. Toward this aim, siRNA oligonucleotides specific for SHIP2 were transfected into HEKs and harvested for Western blot analysis after 72 h. Consistent with our previous experiments, reduced SHIP2 levels resulted in a concomitant increase in phosphorylated AKT (p-Akt) (FIG. 3B).

In view of these observations, we reasoned that increased levels of SHIP2 in HEKs after treatment with Antago-205 might decrease levels of p-Akt and phosphorylated BAD (p-BAD). Western blot analysis was used to measure the protein levels of SHIP2, pan (1/2/5)Akt, p-Akt, BAD, p-BAD, phosphorylated PTEN (p-PTEN), and phosphorylated GSK3β (p-GSK3β) in HEK cells after Antago-205 treatment. We observed an increase in SHIP2 and a coordinated decrease in p-Akt and p-BAD when compared with the irrelevant antagomir or untreated HEKs (FIG. 3A). However, no major change in total Akt, BAD, p-PTEN, or p-GSK3β levels was observed. Moreover, silencing of SHIP2 to prevent its induction by Antago-205 treatment led to an increase in p-Akt (FIG. 3C). Taken together, these studies demonstrate that SHIP2 is regulated by miR-205 and is required for the negative regulation of the Akt pathway in keratinocytes (FIG. 3A).

One of the outcomes of Akt signaling is to induce endogenous BAD phosphorylation, which ultimately leads to the inhibition of BAD-dependent death (29). To address whether the lower levels of p-BAD resulting from the down-regulation of miR-205 (FIG. 3A) would induce keratinocyte apoptosis and cell death, we determined the number of early and late apoptotic keratinocytes after treatment with Antago-205. As expected, there were few early apoptotic cells (1%) in the untreated and irrelevant antagomir-treated (2%) keratinocytes, whereas Antago-205 caused an ≈10-fold increase in early apoptotic cells as judged by annexin V staining (FIG. 3D). Similarly, there was a notable increase in propidium iodide staining, indicating elevated levels of cell death (FIG. 3D). This dramatic increase in apoptosis and cell death indicates that miR-205 may enhance keratinocyte survival by negatively regulating SHIP2.

miR-205 Is Abundant in SCC Cell Lines. It has been reported that miR-205 is overexpressed in head and neck SCC cell lines (30, 31); however, no attempt has been made to validate potential targets of miR-205 in these cell lines. We postulated that if SHIP2 levels are controlled by miR-205, we would see a correlation between miR-205 and SHIP2 in oral SCC cell lines. We cultured SCC9 (tongue (32)), SCC68 (oral (32)), and CAL27 (tongue (33)) cell lines and observed a reciprocal relationship between the miR-205 levels and SHIP2 expression in these cells (FIG. 3E). SCC68 and CAL27, aggressive oral SCC lines (33-35), had high levels of miR-205 and low amounts of SHIP2. SCC9, which is minimally invasive (36) had lower amounts of miR-205 along with higher levels of SHIP2 (FIG. 3E). Immunoblotting of SHIP2 in oral SCCs showing a marked decrease in SHIP2 in SCC68 and CAL27 cells.

Treatment of SCC68 cells with Antago-205 showed (i) a dramatic decrease in miR-205 levels (FIG. 3F), (ii) an increase in SHIP2 expression (FIG. 3G), (iii) a decrease in p-Akt and p-BAD expression (FIG. 3G), and (iv) an increase in apoptotic cells (FIG. 3H) paralleling our observation in normal HEKs (FIG. 3D). Taken together, these results provide additional evidence that SHIP2 levels are regulated by miR-205 and suggest that high levels of miR-205 may contribute to SCC pathogenesis via a SHIP2-mediated enhancement of Akt signaling and cell survival. The restoration of SHIP2 in SCCs via an antagomir to miR-205, which dampens Akt signaling and increases apoptosis, might be a novel use for this antagomir in the treatment of these neoplasias.

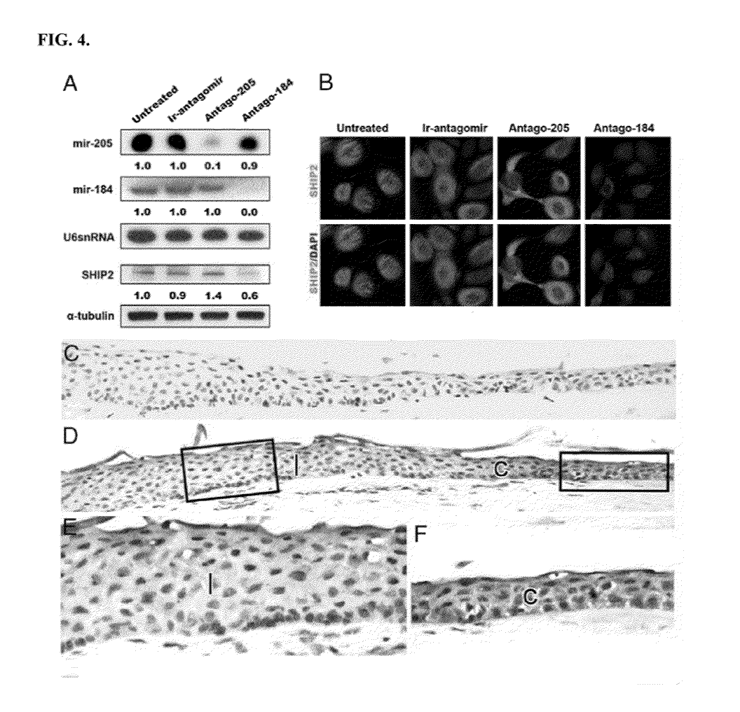

SHIP2 Regulation Is Unique in Corneal Keratinocytes. Having established that SHIP2 is a target of miR-205 in HEKs and SCC cell lines, we next examined the relationship between SHIP2 and miR-205 in human corneal epithelial keratinocytes (HCEKs). The situation in HCEKs is more complex because these cells express miR-184 and miR-205 (FIG. 4A), which interact to maintain SHIP2 levels in HeLa cells (FIGS. 1D and E and 2C). We reasoned that if miR-184 normally maintains SHIP2 levels by inhibiting the interaction of miR-205 with SHIP2, treatment of HCEKs with an antagomir to miR-184 would “release” miR-205 to down-regulate SHIP2. As expected, both SHIP2 expression and miR-184 levels decreased 72 h after treatment with Antago-184 (FIG. 4A and B). In contrast, Antago-205 resulted in a down-regulation of miR-205 and an increase in SHIP2 levels compared with the untreated and control cells (FIG. 4A and B).

Our previous in situ hybridization studies demonstrated that miR-184 was expressed in the corneal epithelium but not in the limbal epithelium, whereas miR-205 was expressed in both the corneal and limbal epithelia (14). If the function of miR-184 in corneal epithelium is to maintain SHIP2 levels by antagonizing miR-205, SHIP2 staining should be more intense in corneal versus limbal epithelium. Indeed, SHIP2 was detected immunohistochemically in normal human corneal epithelium (FIG. 4D and F) whereas much less SHIP2 staining was observed in the limbal region (FIG. 4D and E). These in vivo data strongly supports our in vitro findings that miR-184 antagonizes miR-205 to maintain SHIP2 levels.

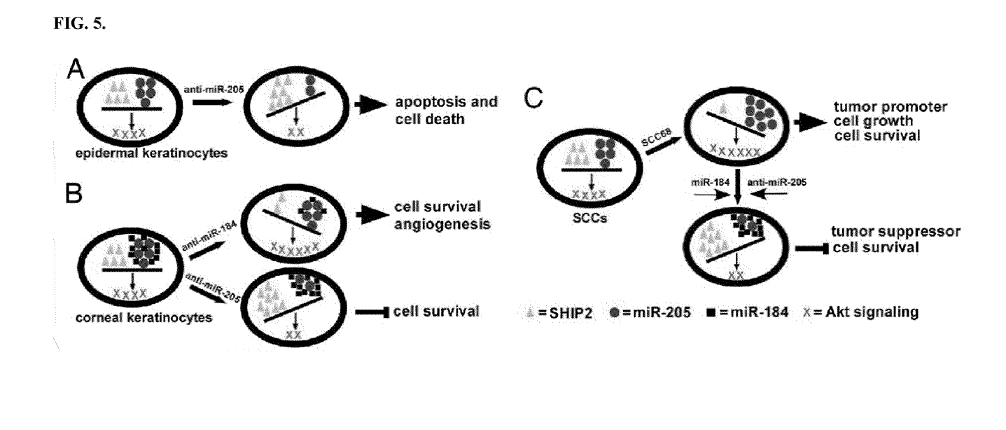

We propose that a balance exists between miR-184 and miR-205 and that this maintains SHIP2 levels (FIG. 5A); however, abrogation of miR-205 elevates SHIP2 because the miR-184/205 balance is altered and miR-184 alone has no inhibitory effect on SHIP2 (FIG. 5B). Similar to the HeLa cell transfections, miR-184 antagonizes miR-205 to maintain SHIP2 levels in corneal keratinocytes and corneal epithelium, and this highlights the uniqueness of the corneal epithelium with respect to SHIP2 regulation (FIGS. 4 and 5B).

Discussion

A chief impediment to understanding miRNA function has been the relative lack of experimentally validated targets. We demonstrate that SHIP2 mRNA is a target of miR-205 in HEKs and that, in HCEKs, miR-184 antagonizes miR-205, thereby maintaining SHIP2 levels. To our knowledge, this is the first example in a vertebrate system where one miRNA abrogates the inhibitory function of another. Our mutation analyses indicate that miR-205 binds to SHIP2 mRNA leading to translational repression. This has been proposed as the “classical” manner in which miRNAs affect protein synthesis in mammalian systems (1). The mechanism by which miR-184 negatively regulates miR-205 is unique. Binding of miR-184 to its seed sequence has no direct effect on SHIP2 translation, but instead prevents miR-205 from interacting with SHIP2 mRNA. This neutralizes the inhibitory activity of miR-205 on SHIP2, a situation special to the corneal epithelium because this is the only known epithelium that exhibits overlapping expression of miR-184 and miR-205 (14). Previously, investigators have considered the regulation of proteins or mRNAs by miRNAs as a one-to-one event; however, our findings indicate that in some instances the situation is more complex and that cross-talk between individual miRNAs can occur.

The need for maintaining SHIP2 levels, which down-regulate the Akt pathway, may relate to the requirement of corneal avascularity so that light required for vision can be transmitted to the lens. Inhibition of Akt can lead to the down-regulation of VEGF, which can repress angiogenesis. We suggest that SHIP2, via its ability to negatively regulate the Akt pathway, could suppress corneal angiogenesis through inhibition of VEGF (37). In this scenario, SHIP2 would be functioning similarly to inhibitory PAS domain protein, which has been shown to maintain an avascular phenotype in corneal epithelium via the negative regulation of VEGF (38).

Despite the ubiquitous distribution of SHIP2 in vertebrate tissues (28, 39) little attention has been directed toward this lipid phosphatase in stratified squamous epithelia, and consequently the function(s) of endogenous SHIP2 in these tissues remain poorly understood. Antago-205 increased keratinocyte SHIP2 levels, which was coordinated with a dampening of Akt signaling (FIG. 5A). Moreover, the down-regulation of miR-205 markedly increased keratinocyte apoptosis and cell death. This is consistent with the report that SHIP2 overexpression in MDCK epithelial cells resulted in cytotoxicity (40). We believe that one of the functions of miR-205, which is broadly expressed in epithelia, is to control SHIP2 levels and maintain cell survival through the Akt pathway.

It is becoming increasingly clear that alterations in miRNAs may adversely impact on cancer (10, 41-43). Of particular relevance to the present study are observations that miR-205 is up-regulated in a variety of carcinomas (8, 9, 30 31, 44, 45). Our finding that elevated levels of miR-205 markedly reduce SHIP2 in aggressive SCC cell lines provides some insight into a potential role of miR-205 in SCCs. PTEN (phosphatase and tensin homologue deleted on chromosome 10) is a lipid phosphatase similar to SHIP2 in that PIP₃ is a common lipid substrate (for review see ref. 46). PTEN is more widely regarded as a tumor suppressor than SHIP2; however, PTEN mutations are rarely found in head and neck, oral, and skin SCCs (47-49), suggestive that another tumor suppressor gene may be associated with the development of these neoplasias (49). Our observations indicate that SHIP2 might fulfill this role through its negative regulation of the Akt pathway, which is frequently deregulated in many types of cancer (for review see ref. 50). Because down-regulation of miR-205 in an aggressive SCC cell line restores SHIP2, we suggest that miR-205 may be viewed as a tumor promoter in the context of SCCs (FIG. 5C). Therefore (i) miR-205 might have diagnostic value in determining the aggressivity of SCCs, and (ii) an antagomir to miR-205 or ectopic expression of miR-184 could be novel therapeutic approaches for treating aggressive SCCs (FIG. 5C).

The idea that SHIP2 might function as a tumor suppressor in keratinocytes makes excellent biological sense from the perspective of corneal epithelial SCCs. These tumors develop from limbal rather than corneal epithelium (51). It is noteworthy that the stem cell compartment, the primary site for malignant transformations (52, 53), is localized to the limbus (15-17). We suggest that an additional factor for a limbal origin of corneal epithelial SCCs may be the absence of miR-184 in the limbal epithelium; because miR-184 is present in the corneal epithelium, this helps preserve SHIP2 levels (FIG. 4D and F) thereby maintaining the presence of a potential tumor suppressor (FIG. 5B). Conversely, the abrupt absence of miR-184 in the limbal epithelium enables miR-205 to negatively regulate SHIP2 levels (FIG. 4D and E), decreasing its potential tumor suppressor function in a stem-cell enriched region. As many neoplasias result from underexpressed tumor suppressor genes, down-regulation of SHIP2 in limbal basal cells could contribute to the neoplastic transformation of these cells.

Materials and Methods

Cell Culture. Primary human epidermal keratinocytes (HEKs) were grown in keratinocyte serum-free media (154 media; Cascade Biologicals Corp.) containing HKGS growth supplements and 70 μM CaCl₂. HCEKs were cultured in CnT20 with supplements (CellnTech Corp.). SCC9 and CAL27 were grown in DMEM/F12 (Gibco Corp.) containing 10% FBS. SCC68 was cultured in Keratinocyte SFM (Gibco Corp.) with recommended supplements. HeLa cells were obtained from American Type Culture Collection and grown in F12 Ham's media with 10% FBS.

Apoptosis Assays. Apoptosis assay was performed on HEKs and the SCC68 cell line 48 h after treatment with either an antagomir directed against miR-205 or an irrelevant antagomir using the Annexin V-FITC Apoptosis Detection Kit 1 (BD Biosciences Corp.) according to the manufacturer's protocols and analyzed by using the FACSCalibur Flow Cytometer (BD Biosciences Corp.).

Constructs and Reagents. A combined luciferase reporter construct containing both miR-184 and mi-R205 consensus target sequences (184/205_PER), which serves as a positive control, was made in pMIR-Report (Ambion Corp.). Top (5′-CTAGTAATATTACCCTTATCAGTTCTCCGTCCCAGACTCCGGTGGAATGAAGGA-3′) and bottom (5′-AGCTTCCTTCATTCCACCGGAGTCTGGGACGGAGAACTGATAAGGGTAATATTA-3′) strand oligonucleotides specifying the 184 target sequence directly followed by the 205 target sequence and containing HinDIII linkers at the 5′ and 3′ ends, respectively, were annealed and ligated to the SpeI and HinDIII sites of pMIR-Report. The 3′ UTR of the human SHIP2 mRNA was generated by RT-PCR and TA cloned into pCR2.1 (Invitrogen Corp.). The SHIP2 3′UTR sequence was verified and was subsequently cloned in between the SpeI and HinDIII sites of pMIR-Report.

Antagomirs directed against miR-184, miR-205, and miR-124 were synthesized by Dharmacon Corp. according to the following structural specification: antagomir-184, 5′-AsCsCsCUUAUCAGUUCUCCGUsCsCsA (SEQ ID NO:7)-Chol-3′; antagomir-205, 5′-CsAsGsACUCCGGUGGAAUGAAsGsGsA (SEQ ID NO:8)-Chol-3′; antagomir-124, 5′-GsGsCsAUUCACCGCGUGCsCsUsU (SEQ ID NO:9)-Chol-3′. Uppercase letters represent 2′OMe-modified nucleotides, “s” represents a phosphorothioate linkage, and “Chol” represents cholesterol.

Immunohistochemistry and Light microscopy. HeLa, HEK, and HCEK cultures grown on glass coverslips were fixed in 4% paraformaldehyde at room temperature for 20 min. After washing in PBS, cells were blocked and permeabilized in PBS containing 2.5% goat serum and 0.1% Triton X-100 at room temperature for 90 min. Cells were incubated with human SHIP2 (1:25; Cell Technologies Corp.) overnight at 4° C. Detection was with Alexa Fluor® 488 goat anti-rabbit IgG (1:500; Invitrogen Corp.) at room temperature for 1 h. As a negative control, antibodies against rabbit IgG were used. Cells were viewed and photographed with a Zeiss UV LSm 510 confocal microscope.

Normal human corneas were obtained from the Illinois Eye Bank. Frozen sections (5 μm) were fixed in 4% paraformaldehyde for 15 min at room temperature. After washing in PBS and blocking PBS containing 2.5% BSA, sections were incubated overnight with SHIP2 (1:500) rabbit polyclonal antibody (ABGENT Corp.) at 4° C. As a negative control, sections were incubated with biotinylated secondary anti-rabbit IgG, avidin-biotin-peroxidase (Vector Corp.), and diaminobenzidine tetrahydro-chloride substrate (Sigma Corp.) Sections were counterstained with hematoxylin.

RNA Isolation and Northern Blots. Total RNA was extracted from cells using TRIzol (Invitrogen Corp.). Total RNA was fractionated on a 15% denaturing (8 M urea) polyacrylamide gel, transferred to nylon membranes (Nytran N; Amersham Biosciences Corp.), and fixed by UV cross-linking. Membranes were probed with ³²P-labeled oligonucleotides complementary to miR-184 or miR-205. Hybridizations were carried out as described previously (1).

Western Blots. HeLa cells, HEKs, SCCs, and HCEKs with mammalian cell lysis Buffer (G-Biosciences Corp.) containing protease (G-Biosciences Corp.) and phosphatase (Calbiochem Corp.) inhibitors. Proteins from total cell lysates were resolved with a 4-20% Tris-HCl gradient gel (Bio-Rad Corp.), transferred to PVDF membranes, blocked in 5% nonfat milk in TBS/Tween 20, and blotted with antibodies for SHIP2 (Cell Signaling Corp.), phosphorylated Akt (Cell Signaling Corp.), Akt (Cell Signaling Corp.), phosphorylated BAD (Cell Signaling Corp.), BAD (Cell Signaling Corp.), phosphorylated PTEN (Cell Signaling Corp.), phosphorylated GSK-3β (Cell Signaling Corp.) and α-tubulin (Invitrogen Corp.).

### REFERENCES

1. Ambros, Nature, 2004, 431:350-355.

2. Lee et al., Cell, 1993, 75:843-854.

3. Esau et al., Journal of Biological Chemistry, 2004, 279:52361-52365.

4. Hornstein et al., Nature, 2005, 438:671-674.

5. Sempere et al., Genome Biology, 2004, 5:R13.11-R13.11.

6. Zhao et al., Nature, 2005, 436:214-220.

7. Leung et al., Cell, 2007, 130:581-585.

8. Iorio et al., Cancer Research, 2005, 65:7065-7070.

9. Iorio et al., Cancer Research, 2007, 67:8699-8707.

10. Lu et al., Nature, 2005, 435:834-838.

11. Ma et al., Nature, 2007, 449:682-688.

12. Yi et al., Nature Genetics, 2006, 38:356-362.

13. Andl et al., Current Biology, 2006, 16:1041-1049.

14. Ryan et al., Molecular Vision, 2006, 12:1175-1184.

15. Cotsarelis et al., Cell, 1989, 57:201-209.

16. Lavker et al., Experimental Eye Research, 2004, 78:433-446.

17. Schermer et al., Journal of Cell Biology, 1986, 103:49-62.

18. Tseng et al., Fortsch Opthalmol, 1991, 88:236-242.

19. Lewis et al., Cell, 2005, 120:15-20.

20. Yi et al., Nature, 2008, 452:225-229.

21. Krek et al., Nature Genetics, 2005, 37:495-500.

22. Barad et al., Genome Research, 2004, 14:2486-2494.

23. Prasad et al., Journal of Biological Chemistry, 2005, 280:13129-13136.

24. Krutzfeldt et al., Nature, 2005, 438:685-689.

25. Lagos-Quintana et al., Current Biology, 2002, 12:735-739.

26. Sasaoka et al., Endocrinology, 2003, 144:4204-4214.

27. Sharrard et al., Cell Signalling, 2007, 19:129-138.

28. Sleeman et al., Nature Medicine, 2005, 11:199-205.

29. Datta et al., Molecular Cell, 2000,6:41-51.

30. Jiang et al., Nucleic Acids Research, 2005, 33:5394-5403.

31. Tran et al., Biochemical and Biophysical Research Communication, 2007, 358:12-17.

32. Rheinwald et al., Cancer Research, 1981, 41:1657-1663.

33. Magne et al., British Journal of Cancer, 2002, 86:1518-1523.

34. Ghosh et al., Cancer, 2002, 95:2524-2533.

35. Sprague et al., Oncology, 2006, 71:282-291.

36. Liu et al., Molecular Carcinogenesis, 2002, 34:131-139.

37. Jiang et al., Proceedings of the National Academy of Science, 2000, 97:1749-1753.

38. Makino et al., Nature, 2001, 414:550-554.

39. Schurmans et al., Genomics, 1999, 62:260-271.

40. Koch et al., Oncogene, 2005, 24:3436-3447.

41. Calin et al., Nature Reviews: Cancer, 2006, 6:857-866.

42. Kumar et al., Nature Genetics, 2007, 39:673-677.

43. Zhang et al., Developmental Biology, 2007, 302:1-12.

44. Gottardo et al., Urologic Oncology, 2007, 25:387-392.

45. Yanaihara et al., Cancer Cell, 2006, 9:189-198.

46. Hawkins et al., Biochemical Society Transactions, 2006, 34:647-662.

47. Chen et al., Oral Oncology, 2000, 36:95-99.

48. Kubo et al., Journal of Dermatological Science, 1999, 19:199-201.

49. Snaddon et al., British Journal of Cancer, 2001, 84:1630-1634.

50. Altomare et al., Oncogene, 2005, 24:7455-7464.

51. Waring et al., American Journal of Opthalmology, 1984, 97:547-559.

52. Miller et al., Biochimica et Biophysica Acta, 2005, 1756:25-52.

53. Perryman et al., Journal of Cellular and Molecular Medicine, 2006, 10:292-308.

54. Hunt et al., Analytical Biochemistry, 2009, 387:1-12.

All publications, patent applications, patents and other references mentioned herein are expressly incorporated by reference in their entirety, to the same extent as if each were incorporated by reference individually. In case of conflict, the present specification, including definitions, will control.

1. A method for diagnosing an aggressive form of squamous cell carcinoma in a patient, the method comprising detecting a level of miRNA-205 in squamous carcinoma cells of the patient wherein the detected level of miRNA-205 in the squamous carcinoma cells of the patient is characteristic of the aggressive form of squamous cell carcinoma, thereby diagnosing the aggressive form of squamous cell carcinoma in the patient.

2. The method of claim 1, further comprising detecting a level of control RNA in the squamous carcinoma cells of the patient and comparing the detected level of miRNA-205 in the squamous carcinoma cells of the patient to the detected level of the control RNA in the squamous carcinoma cells of the patient and calculating a ratio of the detected miRNA-205 to the detected level of control RNA, wherein the ratio is characteristic of the aggressive form of squamous cell carcinoma, thereby diagnosing the aggressive form of squamous cell carcinoma in the patient.

3. The method of claim 1, wherein the level of miRNA-205 is detected by obtaining a nucleic acid sample from the squamous carcinoma cells of the patient and contacting the sample with a probe that binds to miRNA-205.

4. The method of claim 3, wherein the probe is a DNA probe that hybridizes to miRNA-205.

5. The method of claim 3, wherein the probe is an RNA probe that hybridizes to miRNA-205.

6. The method of claim 1, wherein the level of miRNA-205 is detected by performing a solution hybridization assay.

7. The method of claim 1, wherein the aggressive form of squamous cell carcinoma is invasive or has metastasized.

8. The method of claim 1, wherein the aggressive form of squamous cell carcinoma is a tumor that doubles in size over a period of less than about six (6) months.

9. The method of claim 1, wherein the aggressive form of squamous cell carcinoma is a tumor that has a diameter greater than about 1.5 cm.

10. The method of claim 1, wherein the aggressive form of squamous cell carcinoma is a recurring form.

11. A method for treating or preventing an aggressive form of squamous cell carcinoma in a patient in need thereof, the method comprising administering to the patient an inhibitor of miRNA-205.

12. The method of claim 11, wherein the inhibitor is an antagomir of miRNA-205.

13. The method of claim 11, wherein the inhibitor is miRNA-184.

14. The method of claim 11, wherein the inhibitor is administered via expression from an ectopic vector.

15. The method of claim 11, wherein the aggressive form of squamous cell carcinoma is invasive or has metastasized.

16. The method of claim 11, wherein the aggressive form of squamous cell carcinoma is a tumor that doubles in size over a period of less than about six (6) months.

17. The method of claim 11, wherein the aggressive form of squamous cell carcinoma is a tumor that has a diameter greater than about 1.5 cm.

18. The method of claim 11, wherein the aggressive form of squamous cell carcinoma is a recurring form.

19. A method for increasing expression of SHIP-2 in a cell, the method comprising introducing an inhibitor of miRNA-205 to the cell.

20. The method of claim 19, wherein the inhibitor is an antagomir of miRNA-205.


SEQUENCE LISTING (9 sequences)

SEQ ID NO: 1 - RNA, 22 nt, Homo sapiens
uccuucauuccaccggagucug

SEQ ID NO: 2 - RNA, 22 nt, Homo sapiens
uggacggagaacugauaagggu

SEQ ID NO: 3 - RNA, 4737 nt, Homo sapiens
cugaggccggcgcugcaggcagcggcggcugcgcggugaacgaggcggccugcgcggcggagugcugagucccgauccccggcucuguccggcccacggauccucaagcccgggccccgggcccggccccagccucagcccugagcgucucggggcggauggcgcggggcggcgggggcgggcggugcugagcccugcgcgggccauggccucggccugcggggcgccgggcccggggggcgcccugggcagccaggcccccuccugguaccaccgcgaccugagccgggcggccgcggaggagcugcuggcccgggcgggccgcgauggcagcuuccugguccgagacagcgagagcguggcgggggccuucgcgcucugcguccuguaucagaagcaugugcacacguaucgcauucugccugauggagaagauuucuuggcugugcagaccucgcagggugugccugugcgccgcuuccagacccugggugagcucaucggccuguacgcccagcccaaccagggccuugugugcgcccugcuucuuccuguagagggugagcgagagccggacccaccggaugaccgggaugccucagauggggaggaugagaagcccccgcugcccccgcgcucuggcuccaccagcauuucugcccccacugggcccagcaguccccugccagcuccugagacucccacagcuccagcugcugagagugcucccaaugggcugagcaccgucucgcacgacuaccugaaaggcagcuaugggcuggaccuggaagcugugagggguggagccagccaccugccccaccucacccguacccucgcuaccucaugccggaggcugcacagugagguggacaagguccugucaggccuggagauccuguccaagguguuugaccagcagagcucgcccauggugacccgccuuuugcagcagcagaaccugccacagacaggggagcaggaacuagagagccuggugcugaagcugucagugcuaaaggacuuccugucaggcauccagaagaaggcccugaaggcccuacaggacaugagcuccacagcacccccagcuccgcagccauccacacguaaggccaagaccauccccgugcaggccuuugaggugaagcuagaugugacccugggugaccugaccaagauugggaagucacagaaguucacgcugagcguggauguggagggugggcggcuggugcugcugcggagacagcgggacucccaggaggacuggaccaccuucacgcacgaccgcauccgccagcucauuaagucccagcguguccagaacaagcuggguguuguguuugagaaggagaaggaccggacucagcgcaaggacuucaucuuugucagugcccggaagcgggaggccuucugccagcuguugcagcucaugaagaacaagcacuccaagcaggacgagcccgacaugaucucagucuucauaggcaccuggaacaugggaaguguaccaccuccaaaaaacgugacauccugguucacaucgaagggucuggggaagacccuggacgaggucacagugaccauaccccaugacaucuaugucuuugggacccaggagaacucagugggcgaccgcgaguggcuggaccuacugcgcgggggccucaaggagcuuacggaucuggauuaccgcccgauugccaugcaaucacuguggaauaucaagguggcagugcuggucaagccagagcacgagaaccguaucagccaugucaguacguccagugugaagacuggcaucgccaacacccuggggaacaagggggcugugggcgucuccuucauguuuaauggcaccucauuuggcuuugugaauugucaccucaccucgggaaaugagaagacggcucggaggaaccaaaacuacuuggacauccugcggcugcucucgcugggcgaccggcagcucaaugccuuugacaucucucugcguuucacacaccucuucugguuuggggaccucaacuaccgccuggacauggauauccaggagauccugaacuacaucagcaggaaagaguuugagccccuccucaggguggaccagcucaaccuggagcgggagaagcacaaggucuuccuucgauucagugaggaggagaucuccuucccacccaccuaccgcuaugagcgggguucccgggacacauaugccuggcacaagcagaagccaacugggguccggaccaaugugcccucauggugugaccggauucuguggaaauccuacccugaaacucacaucaucugcaauucuuaugguugcacugaugacaucgucaccagcgaccauucccccguguuugggacauuugagguuggaguuaccucccaguucaucuccaagaaagggcucucaaagacuucagaccaggccuacauugaguuugagagcaucgaggccauugugaagacagccagccgcaccaaguucuucaucgaguucuacucuaccugccuggaggaauacaagaagagcuuugagaaugaugcccagagcagugacaacaucaacuuccucaaagugcaguggucuucacgccagcugcccacgcucaaaccaauucuggcugauaucgaguaccugcaggaccagcaccuccugcucacagucaaguccauggauggcuaugaauccuauggggagugugugguugcacucaaauccaugaucggcagcacggcccaacaguuccugaccuuccuaucccaccguggcgaggagacaggcaauaucagaggcuccaugaaggugcgggugcccacggagcgccugggcacccgugagcggcucuacgaguggaucagcauugauaaggaugaggcaggagcaaagagcaaagcccccucugugucccgagggagccaggagcccaggucagggagccgcaagccagccuucacagaggccuccugcccgcucuccagguuauuugaagaaccagagaaaccgccaccaacggggaggcccccagccccaccccgagcagcuccccgggaggagcccuugacccccagguugaagccagagggagcuccugaaccagaagggguggcggcccccccacccaagaacagcuucaauaacccugccuacuacguccuugaaggggucccgcaccagcugcugcccccggagccacccucgccugccagggccccugucccaucugccaccaagaacaaaguggccauuacagugccugcuccacagcuugggcaccaccggcacccucgugugggagaggggaguucuucagaugaggagucuggaggcacacugcccccuccagacuuuccaccuccaccacugccggacucagccaucuuccugccccccagccuggauccuuuaccagggccagugguccggggccgugguggggcugaggcccguggcccaccaccucccaaggcccauccaaggccuccacugcccccaggccccucaccagccagcacuuuccugggggaagugggcaguggggaugaccgguccugcucggugcugcagauggccaagacgcugagcgagguggacuaugccccugcugggccugcacgcucagcgcuccucccaggcccccuggagcugcagcccccccggggacugcccucggacuauggccggccccucagcuucccuccaccccgcauccgggagagcauccaggaagaccuggcagaggaggcuccgugccugcagggcgggcgggccagcgggcugggcgaggcaggcaugagugccuggcugcgggccaucggcuuggagcgcuaugaggagggccuggugcauaauggcugggacgaccuggaguuucucagugacaucaccgaggaggacuuggaggaggcuggggugcaggacccggcucacaagcgccuccuucuggacacccugcagcucagcaagugauagcggaggcaccacgaagcugugaacucagagccccucccugcuaccaaggcccagcuauggccccaggguugaaaaguuaugagggucagggcaguaucucucugccuauuuauuggggugccuauuuauuggggaucugcauuccccgcugcccaaucauuugcaaugcccuaauuagggcauccugccccucgccuuuuaggcucaggacggaaggucaguugccaugguuaccgaggacccugguuacucuggugcuguccuguuuuacuggaccccgccucccagccccaggggugccuguggggguccauuuggguacgucugggcccccacuuucaccaguuucugcggccuuccaccgggccugaaccacagcggaggagcuccgcuaagaccuccccacccccgcugggggugggggcggguguccguccggaaaugaaggaauagcccgaggaccgggcugggguuuauuuaaacuguucugugugggucuggggagggagagcaccuuaauauuauugggguugguugggguggggcaggaucucagccauaaagugccaguuugcuuaguucucacugucuccuggucugugcugcccugcucuggggaugcacggcggcagggugggggagggagguuccucgcaggucucagcccgggacagggucuugcaagcagccuccugggcagucguaaggguugcggcgugaugucuucaauaaauuaaguuuuauuuggaaaa

SEQ ID NO: 4 - RNA, 40 nt, Homo sapiens
ggugggggcggguguccguccggaaaugaaggaauagccc

SEQ ID NO: 5 - DNA, 54 bp, Artificial
  Sense oligonucleotide comprising target sequence for Homo sapiens miRNA-184, target sequence for Homo sapiens miRNA-205, and 5′ and 3′ HinDIII linker sequences
ctagtaatattacccttatcagttctccgtcccagactccggtggaatgaagga

SEQ ID NO: 6 - DNA, 54 bp, Artificial
  Antisense oligonucleotide comprising target sequence for Homo sapiens miRNA-184, target sequence for Homo sapiens miRNA-205, and 5′ and 3′ HinDIII linker sequences
agcttccttcattccaccggagtctgggacggagaactgataagggtaatatta

SEQ ID NO: 7 - RNA, 22 nt, Artificial
  Antagomir directed against Homo sapiens miRNA-184
acccuuaucaguucuccgucca

SEQ ID NO: 8 - RNA, 22 nt, Artificial
  Antagomir directed against Homo sapiens miRNA-205
cagacuccgguggaaugaagga

SEQ ID NO: 9 - RNA, 19 nt, Artificial
  Antagomir directed against Homo sapiens miRNA-124
ggcauucaccgcgugccuu